(19) United States
(12) Patent Application Publication
(10) Pub. No.: US 2001/0036640 A1
(43) Pub. Date: Nov. 1, 2001

(54) System and methods for the high throughput screening of polymorphs

(76) Inventor: Kevin L. D'Amico, Hinsdale, IL (US)

Correspondence Address: Pennie & Edmonds, LLP, 3300 Hillview Avenue, Palo Alto, CA, 94304, US

(21) Appl. No.: 09/840,735

(22) Filed: Apr. 23, 2001

Related U.S. Application Data
(60) Provisional application No. 60/199,396, filed on Apr. 25, 2000.

(51) Int. Cl. G01N 33/53; G01N 23/207; G01N 23/20; G01N 33/543

(52) U.S. Cl. 435/7.1; 378/73; 378/79; 436/518

(57) ABSTRACT

The system includes a synchrotron X-ray source configured to emit an X-ray beam along a beam path. The system also includes a detector, preferably an area detector, such as a CCD detector, disposed in the beam path. The detector is configured to measure diffraction of the X-ray beam caused by a sample. The system additionally includes an automatic sample changer. The automatic sample changer is configured to sequentially position each of plurality of samples into the beam path between the synchrotron X-ray source and the detector. The samples preferably have a mass of about 10 to 100 μgs and are exposed to the X-ray beam for between 5 and 60 seconds before being automatically exchanged with another sample. The method provides that a sample is automatically positioned into an X-ray beam path and irradiated. The diffraction is then detected, the sample removed, and the process repeated for multiple samples.

This application is a continuation-in-part of application Ser. No. 60/199,396 filed on Apr. 25, 2000, entitled “System for the High Throughput Screening of Polymorphs.”

### TECHNICAL FIELD

This invention is directed to an application of powder diffraction X-ray diffractometry. More particularly, the invention is directed to a system and method for rapid screening of small quantities of compounds, typically potential drug candidates, to identify conditions capable of producing new or known polymorph forms of the compounds.

### BACKGROUND OF THE INVENTION

In the pharmaceutical industry there is an ongoing need to identify the crystalline forms of small molecules that are being investigated as potential drug candidates. These molecules typically crystallize in one of several forms, or sometimes a mixture of these forms, that are called polymorphs. See Marcel Dekker, Polymorphism in Pharmaceutical Solids (Drugs and the Pharmaceutical Sciences, Vol 95 H. G. Brittain, ed., New York, 1999). It is important to be able to identify which polymorph form of a molecule is the thermodynamically most stable form, see J. Haleblian and W. McCrone, Journal of Pharmaceutical Science, 58(8), 911 (1969), and which form is being produced by a particular synthetic procedure. It is optimal hat only one form be produced, or at least that if there is a mixture of forms, that the ratio of the forms present in the mixture is predictable.

A powerful way to analyze for the presence of the different crystalline forms is by using X-ray diffraction. See B. E. Warren, X-ray Diffraction, Dover, 1990, for a general treatment of X-ray diffraction. Since the sample that is screened is typically in the form of a collection of randomly oriented small crystallites that together have the form of a finely divided powder, the technique used is called powder diffraction. It is a characteristic of the powder diffraction method that the intensity of the radiation diffracted from the sample has a certain angular pattern. The typical laboratory-based powder diffraction instruments are capable of measuring a powder diffraction pattern from approximately 100-500 mgs of material. The pattern is typically measured by scanning the angle of incidence of the X-ray beam on the sample (typically called theta or omega) and simultaneously scanning a detector that measures the intensity of radiation scattered by the sample as a function of scattering angle (typically referred to as two-theta). This technique gives information about the sample with sensitivity and angular resolution limited by the properties of the X-ray source, the sample, and the detector.

If one sets up a measurement so that the X-ray beam is incident on the sample, and a two-dimensional detector, such as a piece of X-ray sensitive film, is placed downstream of the sample so that the X-ray beam passing through the sample is perpendicular to a detection plane of the detector, then the pattern of scattered radiation appearing on the detector has the form of concentric rings of increasing radius. These are called powder rings and their pattern, intensity, and radii are characteristic of the sample. Therefore an alternative to measuring the pattern by scanning the diffraction angle with a detector is to measure the powder rings with a two-dimensional film-type detector. There are advantages and disadvantages to both of the above described techniques. The advantages and disadvantages depend upon the samples being studied together with other factors.

Since it often happens that several different polymorph forms of a drug candidate molecule are produced during normal processing procedures, it is advantageous to search production conditions as exhaustively as possible to make sure that all possible polymorph forms that could be produced have been identified and characterized. Crystallization conditions can be varied to screen for the presence of different forms, but there are limits on the ability to exhaustively screen a large number of conditions. The practical limitations include the ability to detect the presence of forms in small quantities of materials. A one gram quantity of material may yield at most ten screens of conditions, since a typical X-ray diffraction characterization measurement requires 100-500 mgs of material.

By contrast the ability to detect a sample smaller than a gram of material would permit many more screens to be performed on the same one gram of material. Therefore, a system and method for analyzing and screening small samples of material would be highly desirable.

### SUMMARY OF INVENTION

In one aspect, the invention provides an automated high-throughput system for measuring a large number of powder diffraction patterns from small (typically less than one mg) quantities of material as part of an effort to fingerprint which polymorph form of a potential drug candidate has been produced by a particular synthetic procedure or production protocol. The system includes a synchrotron X-ray source configured to emit an X-ray beam along a beam path. The system also includes a detector, preferably an area detector, such as a CCD detector, disposed in the beam path. The detector is configured to measure diffraction of the X-ray beam caused by a sample. The system additionally includes an automatic sample changer. The automatic sample changer is configured to sequentially position each of plurality of samples into the beam path between the synchrotron X-ray source and the detector.

As will be discussed in more detail in connection with the methods of the invention, below, the diffraction data obtained from the samples need not be of a quality sufficient to solve the structure of the sample compound. Owing to this, and also to the high intensity of the X-ray beam and the rapid rate at which data may be read from the detector, small sample sizes and short irradiation (exposure) times may be utilized, permitting the high throughput analysis of large numbers (e.g., hundreds or even thousands) of samples per day. Generally, the sample size and irradiation time are inversely related, such that, all other things being equal, larger sample sizes require shorter irradiation times and vice versa. Samples capable of generating diffraction data of high enough quality typically need only contain from tens of μgs to 1 mg of compound, and in many embodiments may contain from as little as 50 μg to 500 μg, or even as little as 10 μg to 100 μg, of compound. Irradiation times may be as short as seconds, and will typically range from about 5 to 60 sec, depending upon the size of the sample. In one embodiment of the invention, the samples being analyzed contain from about 10 μg to about 100 μg sample compound and are irradiated with the X-ray beam for about 5 to about 60 sec before being automatically exchanged with another sample.

In another aspect, the invention provides methods for the high throughput analysis of compounds. The methods are particularly suited for analyzing for crystalline form of polymorphs in a high throughput fashion. According to an embodiment of the method, a sample is automatically positioned into an X-ray beam path and irradiated with an X-ray beam produced from a synchrotron X-ray source. The diffraction caused by the diffraction of the X-ray beam by the sample is then detected. The sample is subsequently removed from the X-ray beam path and the process is repeated for multiple samples. The methods may be conveniently carried out using the system of the invention.

The methods may be used for a variety of different purposes, such as screening different synthetic, purification and/or crystallization conditions to determine whether they produce the same or different polymorph forms of particular compound. Alternatively, they may be used to identify or determine synthetic, purification and/or crystallization conditions that yield polymorph forms of a compound that differ from known forms. Such methods are useful in a variety of different contexts, including, for example, identifying synthetic, purification and/or crystallization conditions capable of producing a particular polymorph form of a drug or potential drug, or for identifying conditions capable of generating new polymorph forms of a drug or potential drug as part of an effort to identify more potent or stable forms, etc., of the drug.

The ability to perform such screens does not require that the structures of the sample compounds be elucidated. Different polymorph forms of a compound yield different, unique powder diffraction data and/or patterns, much like different individuals have different, unique signatures and/or fingerprints. Such “fingerprint” or “signature” powder diffraction data and/or patterns may be compared to identify those that are the same or different, indicating which samples comprise the same or different polymorph form of the compound. Thus, according to the methods of the invention, the powder diffraction data and/or patterns of the samples may be compared with one another, or with diffraction data and/or patterns obtained from reference samples, to identify samples that contain a specified, a different, or even a new polymorph form of the compound. The conditions used to generate the samples may then correlated with the information about the polymorph form to identify conditions capable of generating a specified, a different or even a new polymorph form of the compound.

As will be recognized by skilled artisans, because the methods of the invention do not require detailed structural knowledge of each polymorph form produced and analyzed, the X-ray diffraction data obtained for the samples need not be of “structure quality” (i.e., of a quality sufficient to elucidate the absolute structures of the sample compounds). Rather, the data need only be of “fingerprint quality” or “signature quality.” Data are of fingerprint or signature quality if they provide enough information to uniquely define a specific polymorph form of the compound such that different polymorph forms of the compounds may be distinguished from one another. Stated another way, the data need only be of a quality sufficient to provide a unique powder diffraction “fingerprint” or “signature” for each different polymorph form of the compound being analyzed. As a consequence, small samples sizes and short irradiation times may be used, permitting high throughput analysis of large numbers of samples, e.g., on the order of hundreds or even several thousands of samples, per day. Moreover, since small samples sizes may be used, many more samples may be prepared, and hence many more conditions screened, from a given amount of compound. Samples sizes and irradiation times sufficient to generate fingerprint quality diffraction data with a synchrotron X-ray source useful in the methods of the invention are described above. Quite significantly, whereas typical laboratory-based powder diffraction instruments generally require from 100 mg to 500 mg of sample compound, the methods of the invention require only on the order of tens of μgs, typically in the range of about 10 μg to about 100 μg, of sample compound.

Comparisons of diffraction data, whether between the samples or with reference data, may be performed by comparing directly the complete diffraction data, either by visual inspection or with the aid of a computer, or by determining the presence or absence of user-specified “fingerprint” or “signature” features. For example, for each diffraction angle, the user may specify an intensity value (or range of intensity values) characteristic of a particular polymorph form of the compound and screen for those samples that meet the specified criteria. Alternatively, the user may specify intensity ranges for only certain diffraction angles, such as for example, only those angles where intensity peaks are observed. The diffraction data used for the comparison may be the concentric ring powder pattern, an intensity scan of the concentric ring powder pattern, or other data, such as digitized data, obtained from either the powder pattern or intensity scan.

### BRIEF DESCRIPTION OF THE FIGURES

For a better understanding of certain embodiments of the invention, reference should be made to the following detailed description, taken in conjunction with the accompanying drawings, in which:

FIG. 1 is a diagrammatic plan view of a high throughput system for screening for polymorphs according to an embodiment of the invention;

FIG. 2A is a diagrammatic front view of a sample holder and sample changer according to an embodiment of the invention;

FIG. 2B is a diagrammatic side view of the sample holder and sample changer shown in FIG. 2A;

FIGS. 3A-3C are diagrammatic orthographic views of a sample holder according to an embodiment of the invention;

FIG. 4 is a flow chart of a method for the high throughput analysis of crystalline forms of polymorphs, according to an embodiment of the invention;

FIG. 5 shows a powder diffraction pattern from less than 10 μgs of indomethacin recrystallized from 2-butanone;

FIG. 6 shows a powder diffraction pattern from less than 10 μgs of indomethacin recrystallized from chloroform;

FIG. 7 shows a powder diffraction pattern from less than 10 μgs of indomethacin recrystallized from ethyl acetate;

FIG. 8 shows a powder diffraction pattern from less than 10 μgs of indomethacin recrystallized from tetrahydrofuran;

FIG. 9 shows an intensity versus scattering angle plot obtained from the data presented in FIG. 5;

FIG. 10 shows an intensity versus scattering angle plot obtained from the data presented in FIG. 6;

FIG. 11 shows an intensity versus scattering angle plot obtained from the data presented in FIG. 7;

FIG. 12 shows an intensity versus scattering angle plot obtained from the data presented in FIG. 8;

FIG. 13 shows a powder diffraction pattern from a sample of indomethacin as received from the supplier;

FIG. 14 shows a powder diffraction pattern from a sample of indomethacin recrystallized from methanol and water;

FIG. 15 shows an intensity versus scattering angle plot obtained from the data presented in FIG. 13; and

FIG. 16 shows an intensity versus scattering angle plot from the data in FIG. 14 for indomethacin recrystallized from methanol and water.

Like reference numerals refer to corresponding parts throughout the several views of the drawings.

### DETAILED DESCRIPTION OF THE PREFERRED EMBODIMENTS

Certain embodiments of the present invention, discussed in the context of screening for polymorph forms of pharmaceutical compounds, will now be described with reference to the figures.

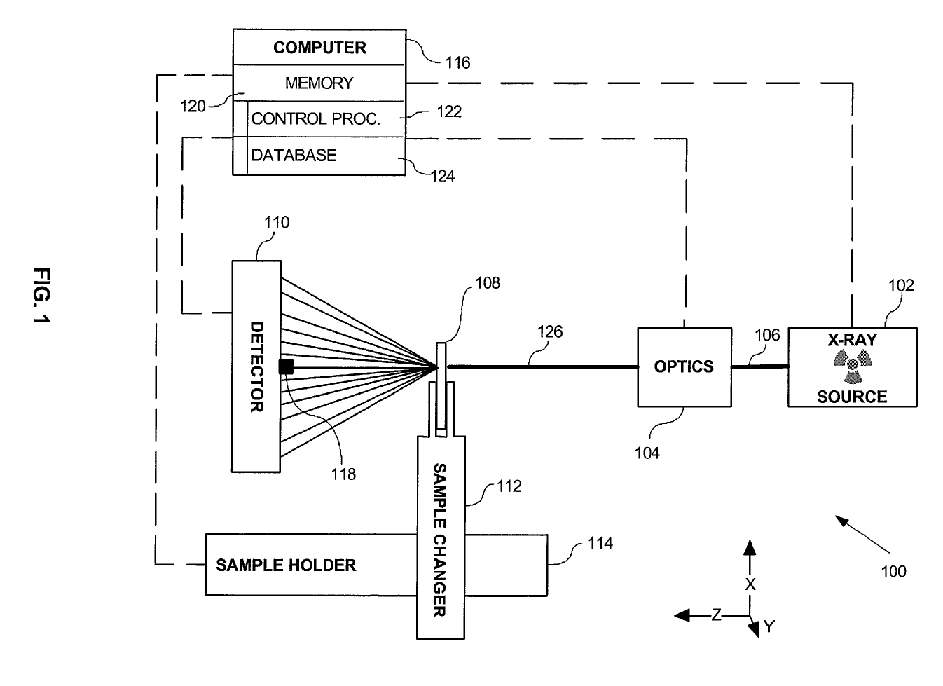

FIG. 1 is a diagrammatic plan view of a high throughput system 100 for screening for polymorphs according to an embodiment of the invention. It should be appreciated that FIG. 1 is merely representational, and is not intended to limit the layout of the high throughput system 100 in any way. For the purpose of describing the figures, the following coordinate system 124 is adopted: the direction of an X-ray beam as it approaches a sample defines a positive Z direction; a positive Y direction is up out of the page; and a positive X direction defines a left handed coordinate system.

Turning to FIG. 1, an X-ray source 102 emits an X-ray beam 106. The properties of the X-ray source 102 that are relevant to this invention are preferably flux, that is number of X-rays delivered to a sample per second; the physical size of the beam spot as it illuminates the sample, expressed as its size in each of the two directions (X and Y) perpendicular to the direction (Z) of propagation of the radiation; energy bandwidth, which is the spread in energy of the X-rays delivered and is usually expressed as a percentage of the energy of the X-rays; and divergence angle, in each of the two directions (X and Y) perpendicular to the direction (Z) of propagation of the radiation. The quality of the diffraction pattern produced by a sample is dependent upon the values of these four parameters. The higher the flux, the more quickly a diffraction pattern can be obtained. The smaller the spot size, the easier it is to measure small quantities of material deposited in a small physical area on a sample tray. The narrower the energy bandwidth, the narrower the diffraction peaks and the easier it is to distinguish neighboring peaks that are close in angle to each other. The higher the collimation, the narrower the diffraction peaks and the easier it is to distinguish neighboring peaks that are close in angle to each other. These physical properties of the beam play off against one another and it is difficult to simultaneously optimize all four parameters. Therefore, for example, if higher flux is obtained at the expense of energy bandwidth, that is, by making the energy bandwidth wider, then the quality of the diffraction pattern will be reduced. If the desired flux is achieved in a small focal spot, the angular divergence is increased, that is by making the radiation striking the sample less parallel in either of the two orthogonal directions (X and Y) perpendicular to the beam propagation direction, then the quality of the diffraction pattern will also be reduced. If it is necessary to make the focal spot large in order to achieve the desired collimation of the beam then the quality of the diffraction pattern will also be reduced. Therefore, in order to achieve a high throughput, the X-ray source 102 must have sufficient flux, a narrow energy bandwidth, a small focal spot size, and high collimation.

Typically, signals observed from a small quantity of a pharmaceutical molecule are weak. To acquire an entire powder pattern in an amount of time that makes high-throughput screening feasible requires that the X-ray source be extremely intense. According to a preferred embodiment of the invention, this period of time is between 5 and 60 seconds. A suitable source of X-rays having the required properties to practice the invention include several synchrotron radiation sources. A synchrotron radiation source is a source of X-rays where the radiation is produced by the accelerating action of a magnetic field on a beam of sub-atomic particles traveling at velocities near the speed of light.

Synchrotron radiation has been used routinely for over two decades in the characterization of materials, including the application of synchrotron radiation to powder diffraction, See J. B. Hastings, W. Thomlinson, and D. E. Cox, “Synchrotron Radiation Research”, H. Winick and S. Doniach, eds., Plenum, New York, (1979); “Synchrotron X-ray Powder Diffraction”, Journal of Applied Crystallography, 17, 85(1984); and D. E. Cox “Powder Diffraction”, in G. S. Brown and D. E. Moncton, eds., “Handbook on Synchrotron Radiation”, Elsevier, 3, 155(1991).

There are several suitable sources of synchrotron radiation that have all the properties necessary to practice this invention. Only those synchrotron radiation sources that produce radiation in the X-ray region of the spectrum are relevant to this invention, i.e., those that produce radiation primarily in the vacuum ultraviolet region of the spectrum.

There are five suitable synchrotron radiation sources in the United States that produce X-rays having sufficient energy to permit the measurements described in this invention to be carried out. These sources are the Advanced Light Source, located in Berkeley, Calif., the Stanford Synchrotron Radiation Laboratory, located in Menlo Park, Calif.; the Cornell High Energy Synchrotron Source, located in Ithaca, N.Y.; the National Synchrotron Light Source, located in Upton, N.Y., and the Advanced Photon Source located in Argonne, Ill. The properties of these sources are compared in G. K. Shenoy, P. J. Viccaro, and D. M. Mills, “Characteristics of the 7-GeV Advanced Photon Source: A Guide for Users,” Document #ANL-88-9, available from Argonne National Laboratory, Argonne, Ill. 60439. Of these, the most suitable source is the Advanced Photon Source (APS). The APS simultaneously has sufficient flux, narrow energy bandwidth, small focal spot size, and high collimation to satisfy a high-throughput screening application according to the invention. Systems utilizing the other radiation sources could achieve the necessary flux at the expense of many or all of the other parameters, making distinguishing subtly different polymorph forms very difficult. Two other sources that are comparable to the APS are available in Japan and France.

Optimum results for the measurements described in this invention are achieved when the beam simultaneously has the highest flux possible, the smallest beam spot possible, the best energy resolution possible, and the best collimation possible. Only in this way can high throughput of samples be achieved with sufficient quality diffraction patterns to distinguish subtle differences between polymorph forms. Preferable values for optimum results are a flux that is at least 2.0E12 X-rays/second; a spot size that is less than about 0.5 mm in diameter (if circular) or has a width of 0.5 mm (if not circular), i.e., approximately 0.5 mm by 0.5 mm; an energy width of at least about 0.02% of the X-ray energy, for example if an X-ray energy of about 12,000 electron volts is used then the energy bandwidth should be less than about 2.5 electron volts; and a collimation that is at most about 0.2 milliradians in each of the two orthogonal directions perpendicular to the beam propagation direction.

The X-ray beam 106 emitted by the X-ray source 102 is conveyed along a beam path to a sample 108 by an optional suitable arrangement of optics 104. The optics 104 condition the X-ray beam by defining the physical dimensions of the beam profile, making the beam 106 sufficiently monochromatic, focusing the beam onto the sample, and blocking stray radiation scattered from the optics 104. The optics 104 may also direct the beam along a chosen beam path 126 at the sample 108. These optics 104 preferably include slits, which define the physical size of the radiation beam; a monochromator, which selects a suitably narrow range of wavelengths centered about a wavelength of approximately 1 angstrom from the synchrotron radiation beam; a mirror, which focuses the radiation to a small spot; additional slits, which further define the size of the beam and reduce unwanted stray background scatter present in the beam after it has struck the mirror, air, or any X-ray transmitting windows after having left the monochromator; lenses; and/or a shutter, which prevents the beam 106 from striking the sample until a detector 110 is ready to detect the diffraction signal. These optics are typical of devices used on general purpose synchrotron radiation beam lines and are commercially available from manufactures like KOHZU Precision Machine Company, Tokyo, Japan. Suitable optics are also discussed in “Synchrotron Radiation Research”, H. Winick and S. Doniach, eds., Plenum, N.Y., (1979).

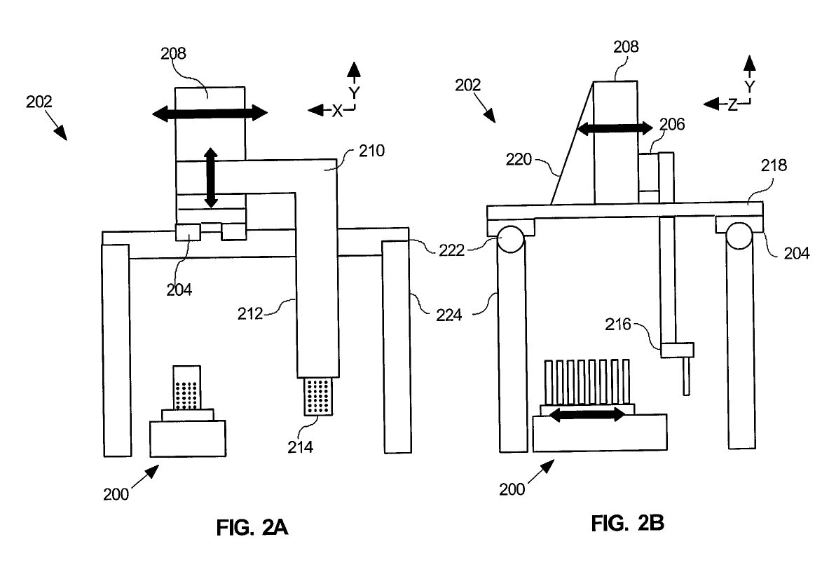

Once X-ray beam 106 has been conditioned by the optics 104, the beam is directed along the beam path at the sample 108. Since the system is designed to process as many samples in as short a time as possible, the samples 108 are preferably automatically changed by a sample changer 112. The sample changer 112 sequentially selects and positions each of a plurality of samples 108 into the beam path. An example of a suitable sample changer is described in U.S. Pat. No. 4,770,593, which is incorporated herein by reference. U.S. Pat. No. 4,770,593 teaches a device where the samples are arranged for data collection on a diffractometer in a reflection geometry and individual samples are taken from a carriage and placed on the diffractometer in succession. The device taught is used for a conventional powder diffractometer based on a standard X-ray tube where the amount of material used must be large, so many hundred mgs must be packed into the sample holder for analysis. However, the disclosed sample changer can be readily adapted for use in the present invention as the sample changer 112. A preferred embodiment of the sample holder 114 and sample changer 112 are shown and described in relation to FIG. 2A and 2B below. Another suitable sample changer is described in copending U.S. application Ser. No. 60/199,396, which is incorporated herein by reference.

Most of the X-ray beam 106 passes directly through the sample 108; however, a certain amount of the X-ray beam is diffracted by the sample. The portion of the X-ray beam that passes through the sample is extremely intense compared to the diffracted X-rays, therefore, this portion of the beam is blocked from reaching detector 110 by a beam stop 118. The detector 110, however, detects the diffracted X-rays as either a concentric ring pattern or as intensity versus scattering angle. Data gathered on the detector 110 is the intensity of the diffracted X-ray beam as a function of X and Y for some fixed displacement in Z of the detector 110 from the sample 108.

Area detectors for powder X-ray diffraction have been used for many years with early measurements being done with X-ray sensitive film. See H. P. Klug and L. E. Alexander, “X-ray diffraction procedures for polycrystalline and amorphous materials” Wiley, N.Y., 1974. Detector technology has evolved from film to two-dimensional wire proportional counters, see S. N. Sulyanov, A. N. Popov, and D. M Kheiker, “Using a two dimensional detector for X-ray powder diffractometry” Journal of Applied Crystallography, 27, 934-942 (1994), and from storage phosphor based image plates, to charge-coupled device based systems. Also, for several years, image plates have been used for powder diffraction systems based on a synchrotron radiation sources. See P. Norby, Journal of Applied Crystallography, 30, 21-30 (1997).

A preferred embodiment of the invention includes a sensitive detector 110 with a fast readout speed such as an area detector based on a charge-coupled device optical sensor, or CCD. Suitable detectors are described in: M. G. Strauss, et al., “CCD-based detector for protein crystallography with synchrotron x-rays” Nuclear Instruments and Methods A297, 275(1990). In addition to their application for protein crystallography, CCD-based detectors have been used with synchrotron radiation sources for time-resolved measurements of phase or chemical changes in solid state systems. See S. O. Svensson, J. Birch, H. Muller, and A. Kvick, “Time-Resolved X-ray Powder Diffraction Using a Large-Area CCD-Based Detector and Rietveld Refinement: Solid State Polymerization of S2N2 to (SN) x” J. Synchrotron Radiation, 4,83-94(1997). The advantage of using a CCD detector is their rapid read out ability relative to the image plate detectors. As described in the above article, CCD detectors have been used with synchrotron radiation sources to repetitively measure the same sample over the duration of a reaction. To date, no one has taught the use of such an area detector for the high throughput analysis of pharmaceutical molecules as disclosed herein. A suitable detector is made by MAR USA, Evanston, Ill.

A computer 116 preferably controls the X-ray source 102, optics 104, sample holder 114, sample changer 114 and/or detector 110. For example, the computer 116 can control the successive positioning of samples into the X-ray beam; control the X-ray source 102, shutter in the optics 104, and detector 110 to simultaneously facilitate the irradiation of each sample 108 for a set amount of time; and store the results of each measurement in a database. The computer 116 preferably includes a memory 120. The memory 120 preferably contains control procedures 122 for controlling the X-ray source 102, optics 104, sample holder 114, sample changer 112 and/or detector 110, and a database 124 for storing data received from the detector 118.

Therefore, the computer system 116 controls the selection of the trays, the positioning of the samples, the acquisition of the data from the detector, and the storage of the analysis data for processing. The diffraction patterns measured can be indexed by standard indexing procedures of powder diffraction data and compared with the patterns acquired from the other samples, with patterns acquired from reference polymorphs or with user-specified data points derived from known polymorph forms. Furthermore, a single computer or multiple computers can be used. Also, the computer can compare the stored data to data from known samples in order to quickly identify a sample.

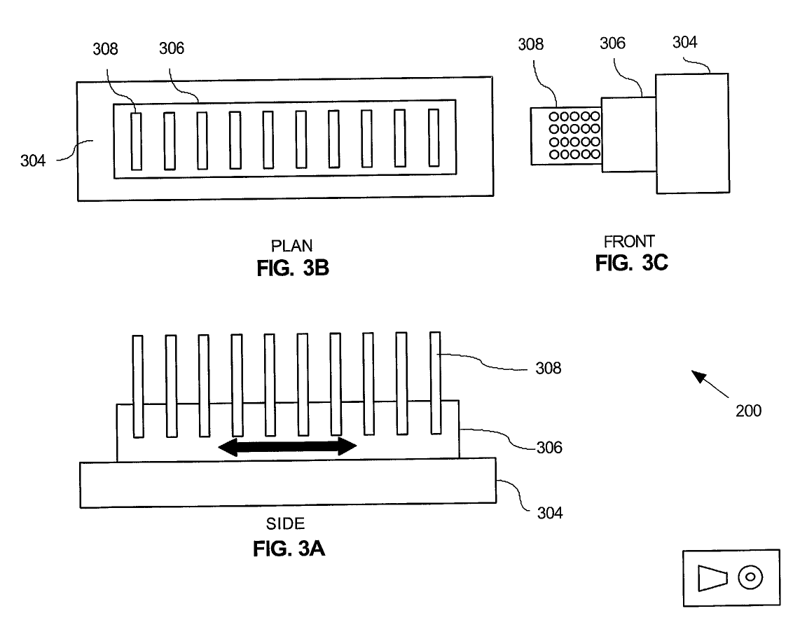

FIGS. 2A and 2B are diagrammatic front and side views, respectively, of a sample holder 200 and sample changer 202 according to an embodiment of the invention. The sample holder 200 is described in further detail in relation to FIG. 3. The sample changer 202 comprises a X-axis linear motion stage 204 movable in the X direction; a Y-axis linear motion stage 206 movable in the Y direction; and a Z-axis linear motion slide 208 movable in the Z direction. Brackets 210 and 212 support the sample tray 214 which is to be analyzed. The sample tray may be nothing more than a substrate, such as a glass slide. A gripping device 216 preferably picks the tray 214 from the sample holder 200. The Z-axis linear motion slide 208 is mounted on a support plate 218 by means of a gusset 220. The Z-axis linear motion slide 208 is mounted to the X-axis linear motion stage 204 which slides on a pair of linear rails 222 mounted onto a set of legs 224.

FIGS. 3A-3C are diagrammatic orthographic views of a sample holder 200 according to an embodiment of the invention. The sample holder 200 comprises a base 304 that holds a carriage 306 that can be moved along the base 304 in the Z direction. The carriage 306 in turn holds multiple sample trays 308 containing samples to be analyzed. The carriage 306 translates the trays 308 in succession so that one tray at a time is placed into a position to be retrieved by the sample changer 202 (FIGS. 2A and 2B). Each tray 308 preferably contains multiple samples mounted on a surface such as a plastic sheet or metal foil that is sufficiently transparent to the wavelength radiation used, which is preferably 1 angstrom. Alternatively, the sample holder 200 can be stationary, while the sample changer, alone, grasps and positions each sample.

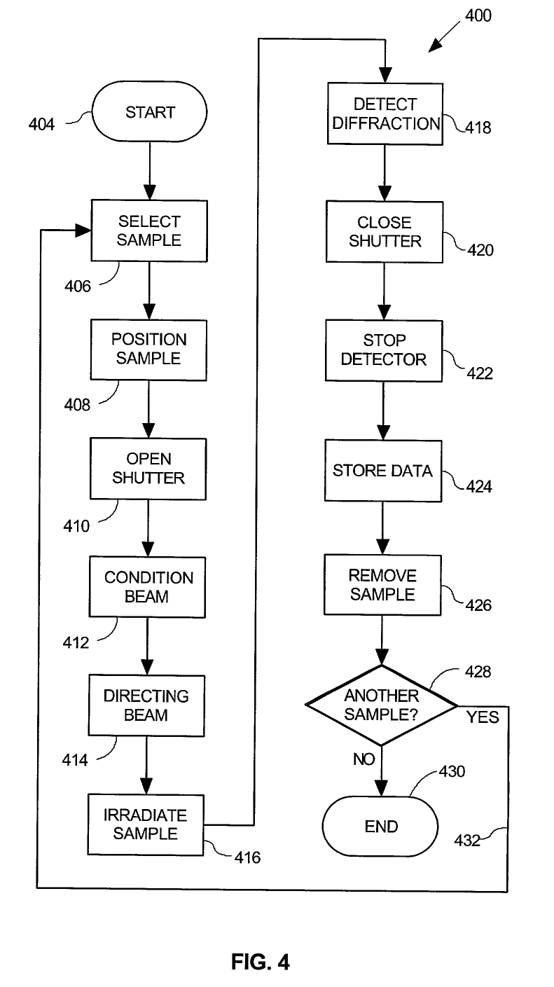

FIG. 4 is a flow chart of a method 400 for the high throughput analysis of crystalline forms of polymorphs, according to an embodiment of the invention. The method is initiated, at 404, and a sample selected, at 406, by the sample changer 112 (FIG. 1). The sample is then automatically positioned, step 408, into an X-ray beam path by the sample changer. The shutter in the optics is then opened, step 410, to allow an X-ray beam to pass through the optics 104 (FIG. 1) to the sample. The optics may also condition, step 412, the X-ray beam, such as by using a collimator or monochromator. The optics may also direct, step 414, the beam at the sample, such as by using mirrors, lenses, or the like.

The sample is then irradiated, step 416, with an X-ray beam produced from a synchrotron X-ray source, preferably for a period of between about 5 to 60 seconds. Diffraction caused by the diffraction of the X-ray beam by the sample, is then detected, step 418, by the detector 110 (FIG. 1). The shutter is then closed, step 420, and the detector stopped, step 422. Data measured by the detector is then stored, step 424, in the database 124 (FIG. 1) of the computer 116 (FIG. 1). The sample is subsequently removed, step 426, from the X-ray beam path.

The system then determines, step 428, whether there is another sample to be detected. If there is another sample to be detected (428—Yes), then the method is repeated, step 432. If there is not another sample to be detected (428—No), then the method ends, step 430.

The system and method may be used in a variety of different contexts. For example, the system and method may be used to identify and/or exhaustively screen synthetic, purification and/or crystallization conditions capable of generating a specific polymorph form, or new polymorph forms, of a compound such as a pharmaceutical drug. When used in this context, a plurality of samples may be generated via different conditions, their diffraction pattern “fingerprints” or “signatures” acquired according to the methods and/or apparatus of the invention, and the fingerprints or signatures compared with fingerprints or signatures of known polymorph forms of the compound to identify those conditions that produces specified or new polymorph forms.

For example, in one embodiment of the invention, a plurality of different crystallization experiments may be carried out with a desired drug. A control crystallization experiment known to produce a specific desired polymorph form of the drug may be carried out as a reference. The samples may be indexed such that they may be correlated with their specific crystallization conditions. Diffraction fingerprints are then acquired for the various different samples, preferably using the high throughput apparatus of the invention. The diffraction fingerprints of the samples are then compared with that of the reference to identify those fingerprints that match. Correlating the matching samples with the crystallization conditions used to generate the samples identifies conditions capable of producing the desired polymorph form of the drug.

Of course, the methods could also be used to identify crystallization conditions capable of producing new polymorph forms of the drug. According to this embodiment, the fingerprints may be compared with reference fingerprints of all known polymorph forms of the drug, and those that differ identified and correlated with their crystallization conditions.

The methods may be used to screen for crystallization conditions capable generating different polymorph forms of a compound even when no information about any polymorph forms are known. According to this embodiment, the diffraction fingerprints of the samples may be compared with one another to identify those that differ. If a complete characterization of these different polymorph forms is desired, larger quantities of the samples may be generated for structure elucidation.

Although certain of the above embodiments describe the acquisition of a reference diffraction fingerprint, those of skill in the art will recognize that while it may be convenient to do so, the reference data need not be obtained along with the sample data. The reference data may constitute previously stored data, or may even be obtained from the literature.

The diffraction fingerprints may be compared visually or with the aid of a computer. The entire dataset may be compared, e.g., the absolute or relative intensities at each diffraction angle, or a subset of the data may be compared, e.g., the absolute or relative intensities at each of a plurality of user-selected diffraction angles. For example, a specific desired polymorph form of a drug may have a characteristic ratio of intensities at two specified diffraction angles. This characteristic ratio, or a user-specified range around this ratio, may be used to screen the sample diffraction fingerprints for those that match. The exact criteria used to determine whether diffraction fingerprints match other diffraction fingerprints will depend upon the particular application and preferences of the user, and will be apparent to those of skill in the art.

In the following examples, conditions were established for observing the diffraction signal from small quantities of samples from a representative pharmaceutical molecule. The examples demonstrate the high throughput capability of a system according to the invention in the context of screening for conditions capable of producing known and new polymorph forms of the representative drug.

### EXAMPLES

Indomethacin has been employed as a test material to illustrate the production and measurement of samples in small quantities. Indomethacin exists in two well-documented polymorph forms, called I, or gamma, and II, or alpha, though others have been reported. See M. O'Brien, J. McCauley, and E. Cohen, “Indomethacin,” in “Analytical Profiles of Drug Substances,” v. 13, p 211-238(1984). A 200 mg quantity was obtained of pure indomethacin from the USP (Rockville, Md., catalog number 34100). The following procedure was used to produce small samples. A known mass of material, typically 2-4 mgs, was placed in a small vial and the appropriate quantity of solvent was added to make a solution of a concentration of 4 mgs in 1 milliliter of solvent. The solvents used were 2-butanone, chloroform, ethyl acetate, and tetrahydrofuran. A known volume of each of these solutions, approximately 5 microliters, was pipetted onto the surface of a glass microscope slide. These solutions each wetted the surface of the glass so that such a drop spread to an approximately 8-10 mm diameter spot. As the solvent evaporated solid material was observed crystallizing at the perimeter of the spot. This material was relatively uniformly distributed at the edge of the spot. A small quantity of this solid was retrieved by scooping it into the end of a standard 0.3 mm diameter Debye-Scherrer capillary tube (obtained from Charles Supper Company, Natick, Mass.). It was estimated that in each case not more than half of the material that solidified at the edge of the drop was retrieved; this is an upper limit. The capillary tubes were mounted at the sample position of a MAR USA detector system, described above, and exposed to the beam produced by the Sector 32 insertion device beam line at the Advanced Photon Source at Argonne National Laboratory.

Each sample was exposed to the beam for 5 seconds. The samples were not rotated or translated during the exposure. The energy of the X-ray beam was 12398.5±0.3 electron volts; the energy width was 0.8 electron volts. This corresponds to a wavelength of 1.00000+/−0.00002 Angstroms. The flux of X-rays on the sample was 2×10E12 per second in an area 0.3 mms high by 0.3 mms wide. The angular collimation of the beam was 0.17 milliradians in the horizontal direction and 0.02 milliradians in the vertical direction.

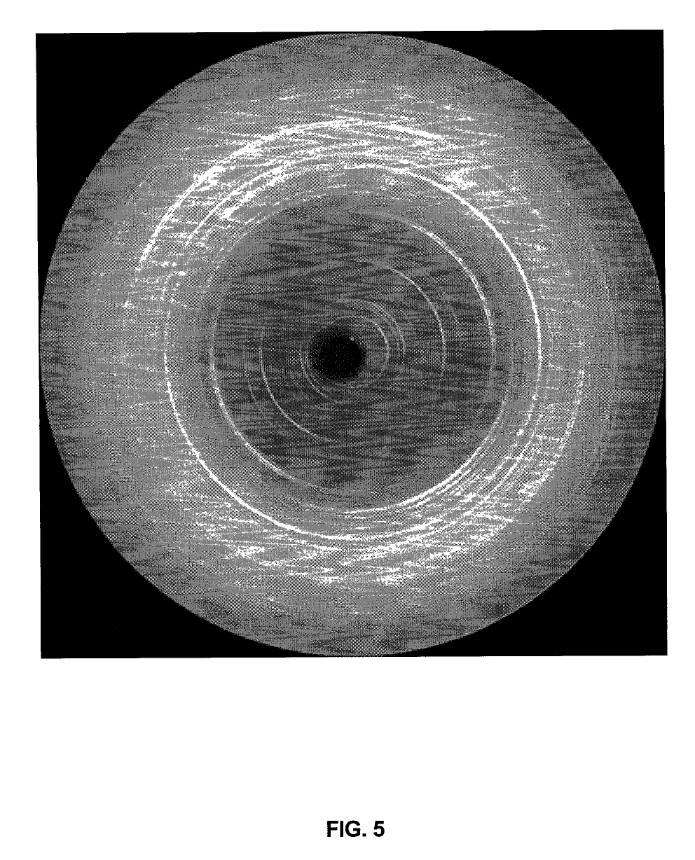

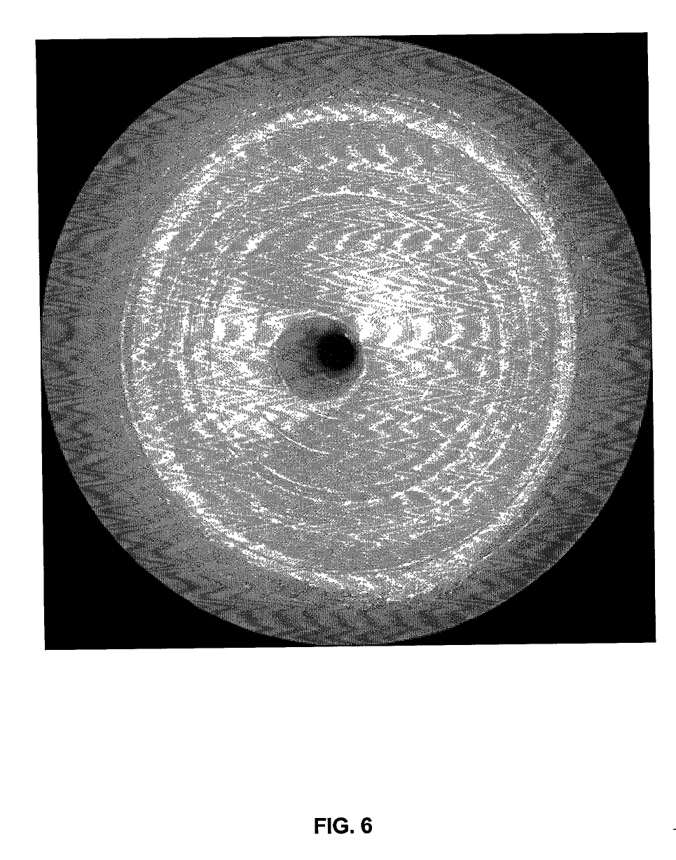

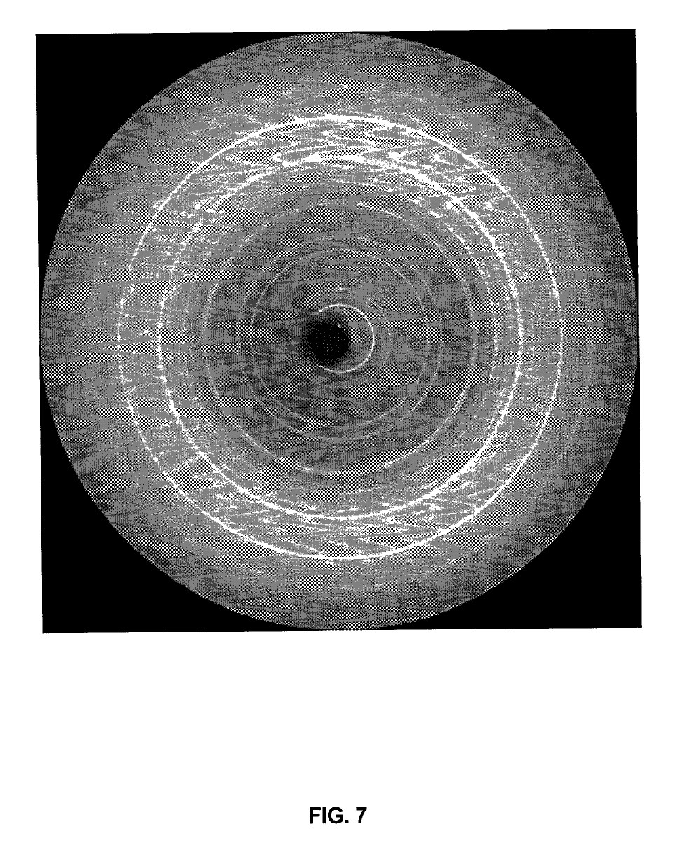

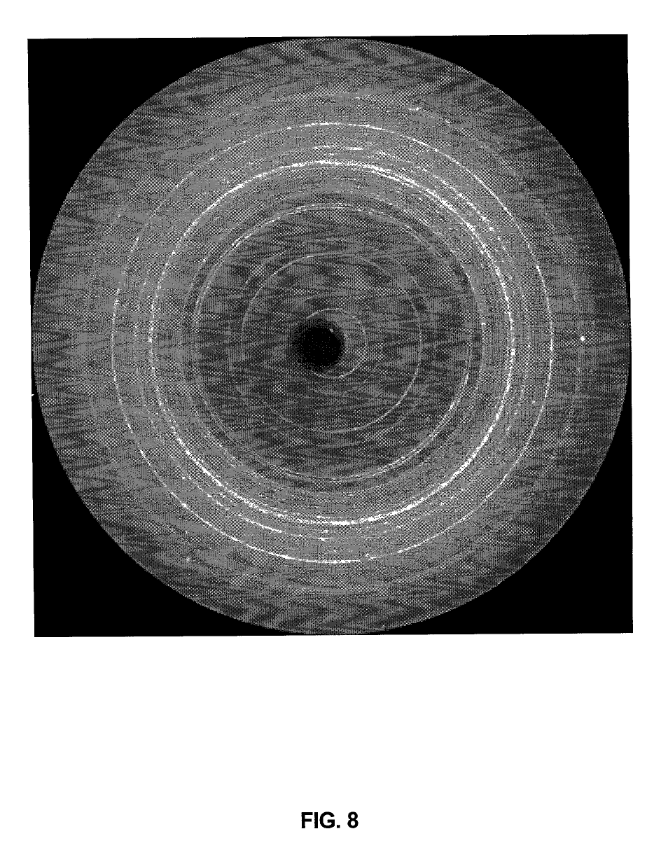

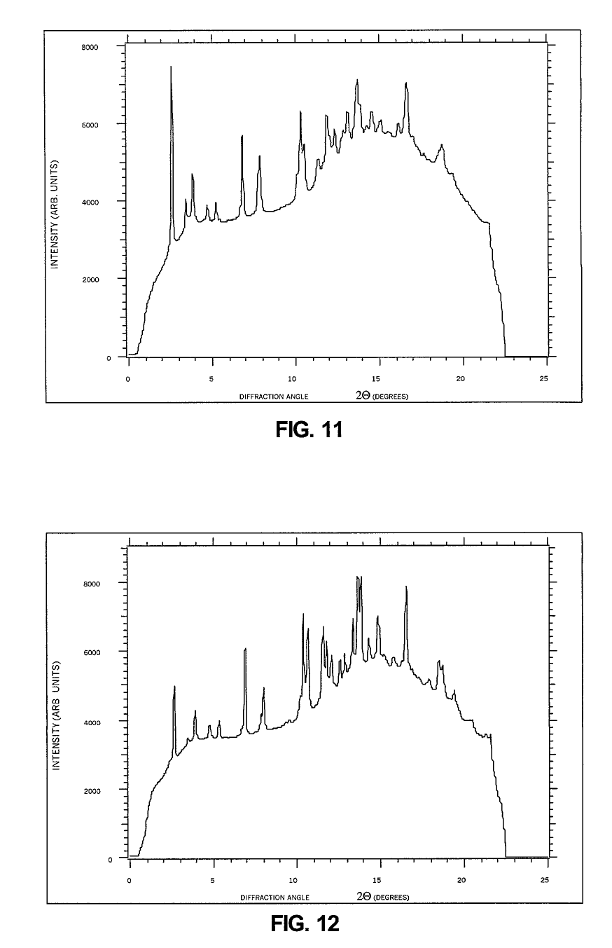

The results are shown in FIGS. 5-12 in two ways: As full images from the detector and as plots of intensity versus scattering angle. FIGS. 5-8 show the images from the detector. In these gray scale images detected X-rays are shown as light parts of the image and lack of detected X-rays are shown as dark parts of the image. The images look similar in that all three have a dark spot in the center which is the shadow of the beam stop located between the sample and the detector. The concentric rings of the powder pattern are clearly shown.

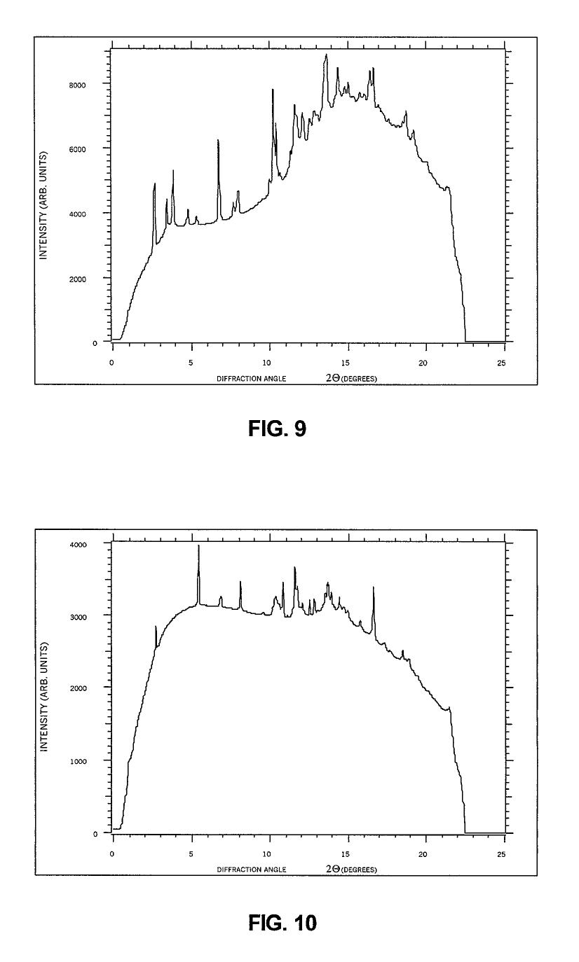

FIGS. 9-12 show a standard intensity versus scattering angle plot. These intensity plots were produced in the following way. The direct beam's position on each of the images in FIGS. 5-8 was determined by locating the center of the concentric rings of the diffraction pattern. The radius of each pixel of the image was determined relative to that center position. The signal for each pixel of a given radius was added to give a total signal at that radius, and that total was divided by the total number of pixels at that radius. This gives the average intensity per pixel at a given radius in pixels. This radius in pixels was converted into a scattering angle in degrees given that the sample to detector distance was 200 mms; each pixel is a square 0.0791 mm on an edge. Averaging over the intensity in all the pixels at a radius is an alternative to merely taking a “cut” through the ring pattern along a single column of pixels, thereby fully using all the data on the image. Note that the images are only displayed with 8 bit depth whereas the line scans show the actual value of the pixel's digitized signal which is a depth of 16 bits. In all cases the intensity versus scattering angle plots show background scatter from both the air in the beam path, at low angles, and a “hump” from the glass of the capillary tube at about 15 degrees or so. In both cases this was verified by looking at air only, with no capillary, and an empty capillary.

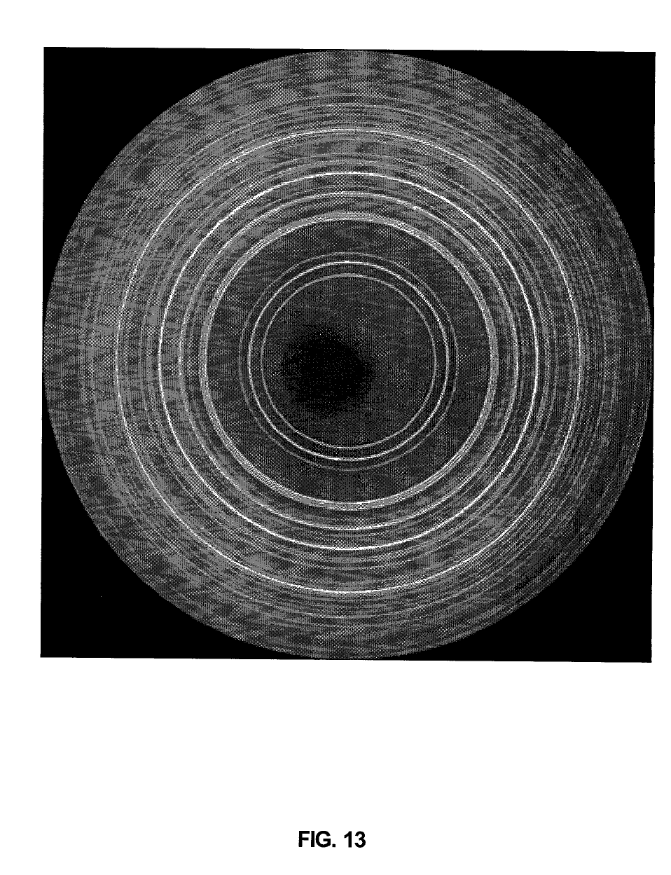

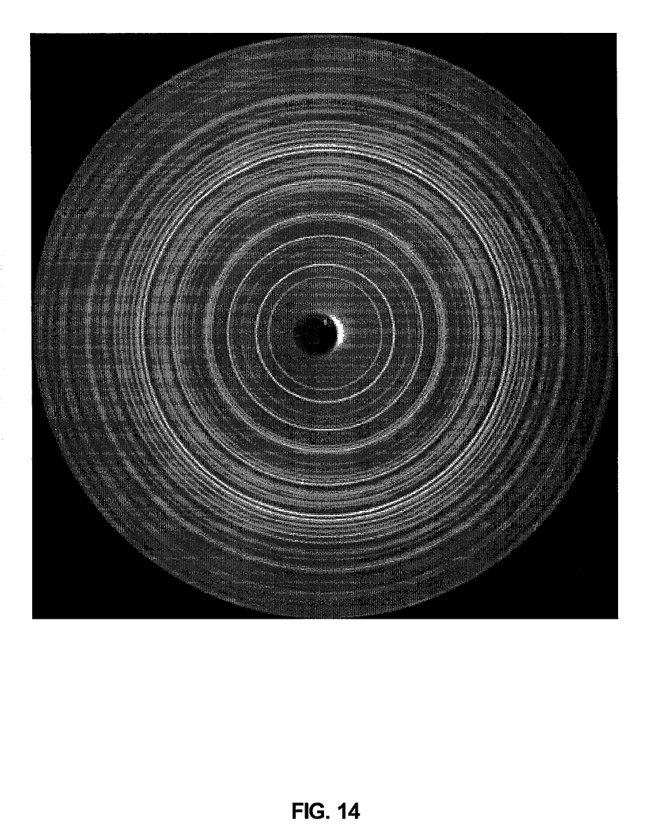

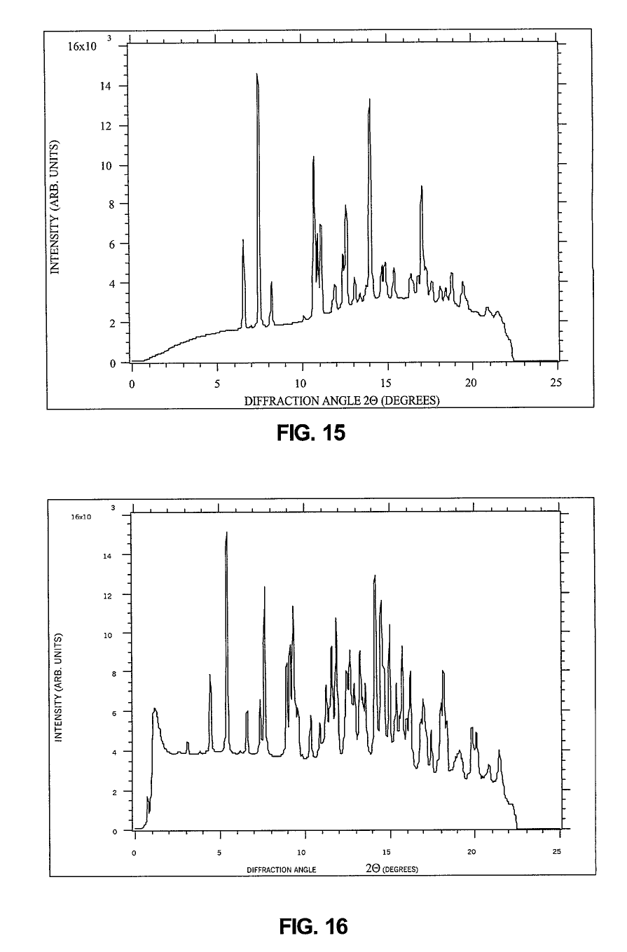

For comparison with known forms the full detector images in FIGS. 13 and 14 are shown for the known forms of indomethacin termed I, or gamma, and II, or alpha, respectively, in M. O'Brien, J. McCauley, and E. Cohen, “Indomethacin,” in “Analytical Profiles of Drug Substances,” v. 13, p 211-238(1984), and the corresponding scattering patterns in FIGS. 15 and 16. The material as received from USP showed the pattern in FIGS. 13 and 15 and corresponds to Form I or gamma. The pattern in FIGS. 14 and 16 is from a sample which was produced by recrystallizing from a water/methanol mixture and corresponds to Form II or alpha.

Clearly the patterns obtained from the four solvents do not correspond to those obtained from either known form, and indeed do not correspond identically to one another. The identity of the forms represented by the patterns is not essential for this analysis. The experiment clearly demonstrates that μg quantities of material can be observed and generate fingerprint quality data sufficient to distinguish different polymorph forms from one another.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, with other elements, materials, and components, and the method performed in a different order, without departing from the spirit or essential characteristics thereof. The presently disclosed embodiments are therefore to be considered in all respects as illustrative and not restrictive, the scope of the invention being indicated by the appended claims, and not limited to the foregoing description.

What is claimed is:

1. A system for the high throughput analysis of polymorph forms of a compound, comprising:
a synchrotron X-ray source configured to emit an X-ray beam along a beam path;
a detector disposed in said beam path, said detector configured to measure diffraction of the X-ray beam caused by a sample; and
an automatic sample changer configured to sequentially position each of a plurality of samples into said beam path between said synchrotron X-ray source and said detector.

2. The system of claim 1, further comprising optics disposed at least partly in said beam path between said synchrotron X-ray source and said automatic sample changer.

3. The system of claim 2, wherein said optics are selected from the group consisting of a monochromator, a collimator, a focusing mirror, slits and a shutter.

4. The system of claim 2, further comprising a computer that controls the system.

5. The system of claim 4, wherein said computer comprises control procedures for controlling the timing of said X-ray source, said optics, and said detector.

6. The system of claim 4, wherein said computer comprises a database for storing diffraction data received from said detector.

7. The system of claim 1, further including a sample holder that holds a plurality of samples.

8. The system of claim 7, wherein the samples are disposed in a plurality of trays and each tray comprises a plurality of samples.

9. The system of claim 7 in which the sample holder is an X-ray transparent substrate and the samples are arrayed on the substrate.

10. The system of claim 7, wherein the samples comprise small organic compounds.

11. The system of claim 7, wherein the sample comprises an amount of compound sufficient to generate a fingerprint quality diffraction pattern when illuminated with the X-ray beam for a duration of about 5-60 sec.

12. The system of claim 7, wherein each sample comprises about 10 μg to about 1 mg of compound.

13. The system of claim 7, wherein each sample comprises about 10 μg to about 100 μg of compound.

14. The system of claim 1, wherein said detector is an area detector.

15. The system of claim 1, wherein said detector is a charge coupled device.

16. The system of claim 1, wherein said sample changer can translate a sample along three axes.

17. A method for the high throughput analysis of polymorph forms of a compound, comprising the steps of:
automatically positioning a sample into an X-ray beam path;
irradiating said sample with an X-ray beam produced from a synchrotron X-ray source;
detecting diffraction caused by the diffraction of the X-ray beam by the sample;
removing said sample from the X-ray beam path; and
repeating said positioning, irradiating, and detecting steps for multiple samples.

18. The method of claim 17, in which the sample and the irradiating step are of a size and a duration, respectively, sufficient to generate a finger-print quality diffraction powder pattern of the sample.

19. The method of claim 17, in which the sample positioned into the X-ray beam comprises about 10 μg to about 100 μg of compound.

20. The method of claim 17, further comprising, after said positioning step, the step of opening a shutter to allow an X-ray beam to pass.

21. The method of claim 17, further comprising, after said positioning step, the step of conditioning the X-ray beam using optics.

22. The method of claim 17, further comprising, after said positioning step, the step of directing the X-ray beam at the sample using optics.

23. The method of claim 17, wherein said irradiating step comprises irradiating said sample for a period of between about 5 to 60 seconds.

24. The method of claim 17, further comprising, after said detecting step, the step of closing a shutter.

25. The method of claim 17, further comprising, after said detecting step, the step of stopping further detection.

26. The method of claim 17, further comprising, after said detecting step, the step of storing diffraction data.

27. A method for identifying conditions capable of generating a specific polymorph form of a compound, comprising the steps of:
acquiring diffraction fingerprints for a plurality of different samples of the compound with the system of claim 1; and
identifying those sample diffraction fingerprints that match a reference diffraction fingerprint of the specific polymorph form of the compound, thereby identifying the conditions capable of generating the specific polymorph form of the compound.

28. A method for identifying conditions capable of generating new polymorph forms of a compound, comprising the steps of:
acquiring diffraction fingerprints for a plurality of different samples of the compound with the system of claim 1; and
identifying those sample diffraction fingerprints that differ from reference diffraction fingerprints of known polymorph forms of the compound, thereby identifying the conditions capable of generating new polymorph forms of the compound.

29. A method for identifying conditions capable of generating different polymorph forms of a compound, comprising the steps of:
acquiring diffraction fingerprints for a plurality of different samples of the compound with the system of claim 1; and
identifying those sample diffraction fingerprints that differ from one another, thereby identifying the conditions capable of generating different polymorph forms of the compound.
